(19) United States
(12) Patent Application Publication
(10) Pub. No.: US 2024/0099651 A1
(43) Pub. Date: Mar. 28, 2024

(54) METHOD FOR PREDICTING PELVIC SAGITTAL BALANCE STATE AFTER HIP REPLACEMENT SURGERY

(71) Applicant: BEIJING JISHUITAN HOSTITAL, Beijing (CN)

(72) Inventors: Yixin ZHOU, Beijing (CN); Hao TANG, Beijing (CN)

(21) Appl. No.: 18/257,149

(22) PCT Filed: Nov. 30, 2020
(86) PCT No.: PCT/CN2020/132566

§ 371 Date: Jun. 13, 2023

(30) Foreign Application Priority Data
Nov. 19, 2020 (CN) 202011299773.6

(51) Int. Cl. A61B 5/00; A61B 5/107; A61B 5/11; A61F 2/46

(52) U.S. Cl. CPC A61B 5/4571; A61B 5/1071; A61B 5/1116; A61B 5/1128; A61B 5/4519; A61B 5/4884; A61B 5/7264; A61B 5/7278; A61F 2/4609; A61F 2002/4633

(57) ABSTRACT

A method for predicting sagittal balance state of pelvis after hip replacement surgery includes obtaining evaluation imaging data of full body sagittal posture of a patient before surgery under different body postures, and obtaining fixed flexion deformity information of bilateral hip joints and an evaluation result of functionality of hip extension muscle clusters through physical examination. The method includes performing posture analysis on the evaluation imaging data of full body sagittal posture under different body postures obtained to obtain analysis results of degree of sagittal imbalance and severity of hip abduction deformity of the patient before surgery. The method further includes predicting pelvic posture rebalance state in a sagittal plane of the patient after hip replacement surgery according to the analysis results of the degree of sagittal imbalance the severity of hip abduction deformity, and the evaluation result of functionality of hip extension muscle clusters of the patient before surgery.

### FIELD OF THE INVENTION

The present disclosure relates to a method for predicting the sagittal balance state of the pelvis, in particular to a method for predicting the sagittal balance state of the pelvis after hip replacement surgery.

### BACKGROUND OF THE INVENTION

A sagittal posture has a significant impact on a pelvic posterior tilt posture after artificial hip joint surgery, and the latter in turn determines a functional orientation of an acetabular cup prosthesis, which is closely correlated to complications such as impact, stress concentration, prosthesis wear, and joint dislocation. When a traditional method is used to evaluate the sagittal posture of a patient before surgery, it is easy to cause problems that:
1) concerning a patient suffering from sagittal imbalance, it is impossible to quantitatively predict the postoperative sagittal balance state of full body and postoperative pelvic posture;
2) it is impossible to quantitatively predict a postoperative spacial position of the cup;
3) it is difficult to locate, according to a functional angle, the orientation of the cup in total hip replacement surgery;
4) it is prone to complications after the rebalance, such as hip joint stress concentration, impact, dislocation, and prosthesis loosening.

### SUMMARY OF THE INVENTION

The objectives of the present disclosure are to provide a method for predicting the sagittal balance state of the pelvis after hip replacement surgery, which can accurately predict the sagittal balance state of the pelvis after hip replacement surgery, and provide a reference basis for planning a target implantation angle of a cup prosthesis in total hip replacement surgery.

To achieve the above objectives, the present disclosure adopts the following technical solution. A method for predicting the sagittal balance state of the pelvis after hip replacement surgery, comprising steps of:
1) obtaining evaluation imaging data of full body sagittal posture of a patient before surgery under different body postures, and obtaining fixed flexion deformity information of bilateral hip joints and an evaluation result of functionality of hip extension muscle clusters through physical examination;
2) performing posture analysis on the obtained evaluation imaging data of full body sagittal posture under different body postures to obtain analysis results of degree of sagittal imbalance and severity of hip abduction deformity of the patient before surgery; and
3) predicting pelvic posture rebalance state in a sagittal plane of the patient after hip replacement surgery according to the analysis results of the degree of sagittal imbalance and the severity of hip abduction deformity as well as the evaluation result of functionality of hip extension muscle clusters of the patient before surgery.

Further, in step 1), when obtaining the evaluation imaging data of the full body sagittal posture of the patient before surgery under different body postures, the different body postures obtained include standing on both legs, standing on one leg, sitting, and squatting.

Further, in step 1), the evaluation imaging data of full body sagittal posture under each body posture at least includes imaging data from the cervical spine to the middle femur.

Further, in step 2), performing posture analysis on the obtained evaluation imaging data of full body sagittal posture under different body postures comprises steps of:
2.1) measuring angle and distance parameters in the evaluation imaging data of full body sagittal posture under different body postures, the angle and distance parameters including pelvic tilting, pelvic incidence, lumbar lordosis, sacral vertical axis, and sacral pelvic angle; and
2.2) performing, based on the parameters measured and standard reference ranges, an analysis of the sagittal balance state of the patient before surgery to obtain the analysis results of the degree of sagittal imbalance and the severity of hip abduction deformity of the patient before surgery.

Further, in step 3), predicting the pelvic posture rebalance state in a sagittal plane of the patient after hip replacement surgery comprises steps of:
3.1) classifying, based on the analysis result of the degree of sagittal imbalance, the evaluation result of the hip extension muscle clusters and the fixed hip flexion deformity information, patients into standing sagittal balance patients, hip extension muscle cluster weakness patients, standing sagittal lumbar vertebra hyperextension patients, and standing sagittal imbalance patients; and
3.2) predicting, for different types of patients, the pelvic posture rebalance state in the sagittal plane of the patients after hip replacement surgery.

Further, in step 3.2), predicting, for different types of patients, the pelvic posture rebalance state on the sagittal plane of the patients after hip replacement surgery comprises:
\textcircled{1} for the standing sagittal balance patients who meet 0 mm<SVA<50 mm, standing PT angle<20°, −10°<PI−LL<10° simultaneously, a change of pelvic posterior tilt angle of the patients after hip replacement surgery that go through pelvic rebalance is 0;
\textcircled{2} for the hip extension muscle cluster weakness patients whose muscle strength<Level 4, a change of pelvic posterior tilt angle of the patients after hip replacement surgery that go through pelvic rebalance is 0;
\textcircled{3} for the standing sagittal lumbar vertebra hyperextension patients whose PI−LL<−10° and SVA<0, in a case that:
A. if fixed flexion deformity of the hip joint <10° and there is any one of situations of severe degeneration/spontaneous fusion/surgical fusion in the lumbar vertebra, then a change of pelvic posterior tilt angle of the patients after hip replacement surgery that go through pelvic rebalance is 0; and
B. if fixed flexion deformity of the hip joint >10° and there is no severe degeneration/spontaneous fusion/surgical fusion in the lumbar vertebras, then a change of pelvic posterior tilt angle of the patients after hip replacement surgery that go through pelvic rebalance is 0.25×PI; and
\textcircled{4} for the standing sagittal imbalance patients with standing SVA>50 mm, in a standing lateral view of the spine-pelvis or full body of the patients, by using a midpoint of a line connecting centers of the bilateral hip joints as a rotation axis and plotting a vertical in accordance to the standard of enabling balance state of the sagittal plane after the surgery, calculating and obtaining a rotation angle α by which a center of T1 vertebra rotates to the vertical to serve it as a change of pelvic posterior tilt angle of the patients after hip replacement surgery that go through pelvis rebalance.

By using the above technical solution, the present disclosure has the advantages of 1. By obtaining the evaluation imaging data of full body sagittal posture of a patient under different body postures, as well as fixed flexion deformity information of the bilateral hip joints and an evaluation result of hip extension muscle clusters, comprehensive analysis is performed on the degree of sagittal imbalance of the patient before surgery, and prediction for postoperative pelvic sagittal posture can be implemented for different types of patients based on results of the analysis so that a prediction result for the patient can be more accurate. 2. Based on the evaluation imaging data of the patient, it can accurately determine the pelvic posture after total hip replacement surgery, and provide the total hip replacement with an accurate reference basis for planning the functional cup angle surgery, thereby optimizing the kinematics dynamics effect of hip replacement, avoiding dysfunction of the prosthesis position due to rebalancing of pelvic posture, and reducing the risk of complications such as impact, dislocation, wear, loosening, etc. Therefore, the present disclosure can be widely applied in the field of predicting the postoperative sagittal balance state of the pelvis.

### DETAILED DESCRIPTION OF THE EMBODIMENTS

The present disclosure will be described in detail in conjunction with the embodiments below.

The present disclosure provides a method for predicting the sagittal balance state of the pelvis after hip replacement surgery, comprising the following steps 1) to 3).
1) Obtaining evaluation imaging data of full body sagittal posture of a patient before surgery under his/her different body postures, obtaining fixed flexion deformity information of bilateral hip joints, and an evaluation result of functionality of hip extension muscle clusters through physical examination.
2) Performing posture analysis on the obtained evaluation imaging data of full body sagittal posture under different body postures to obtain analysis results of the degree of sagittal imbalance and the severity of hip abduction deformity of the patient before surgery.
3) Predicting pelvic posture rebalance state in a sagittal plane of the patient after hip replacement surgery according to the analysis results of the degree of sagittal imbalance and the severity of hip abduction deformity as well as the evaluation result of functionality of hip extension muscle clusters of the patient before surgery.

In step 1), when obtaining the evaluation imaging data of full body sagittal posture of the patient before surgery under different body postures, said different body postures obtained include, but are not limited to, standing on both legs, standing on one leg, sitting, squatting, and the like. In this case, the evaluation imaging data for each body posture should at least include imaging data from the cervical spine to the middle femur.

In step 2), when performing posture analysis on the obtained evaluation imaging data of full body sagittal posture under different body postures, specific steps 2.1) to 2.2) are included.
2.1) Measuring, based on the obtained evaluation imaging data, and relevant angles, distances, and other parameters including pelvic tilting (PT), pelvic incidence (PI), lumbar lordosis (LL), the sacral vertical axis (SVA), and sacral pelvic angle (SPA);
2.2) Evaluating the sagittal balance state of the patient before surgery based on the obtained measurement parameters and standard reference ranges to obtain the degree of sagittal imbalance and the severity of hip abduction deformity of the patient before surgery.

In this case, said standard reference ranges may be the routine ranges that are known to a person skilled in the art, for example standing sagittal imbalance index: SVA (>50 mm being deemed as imbalance), PI−LL difference (>10° being deemed as imbalance), standing PT angle (>20° being deemed as imbalance); hip abduction deformity index, including CL, TK, LL, KF, which will not be described any further herein.

In step 3), predicting pelvic posture rebalance state in the sagittal plane of the patient after hip replacement surgery according to the analysis results of the degree of sagittal imbalance and the severity of hip abduction deformity as well as the evaluation result of functionality of hip extension muscle clusters of the patient before surgery includes the following steps 3.1) to 3.2).
3.1) Based on the evaluation results of the degree of sagittal imbalance, the hip extension muscle clusters, and the fixed hip flexion deformity, patients are classified into standing sagittal balance patients, hip extension muscle cluster weakness patients, standing sagittal lumbar vertebra hyperextension patients, and standing sagittal imbalance patients.
3.2) For different types of patients, prediction is performed on the pelvic posture rebalance state in the sagittal plane of the patients after hip replacement surgery. Specifically,
\textcircled{1} for the standing sagittal balance patients (who meet 0 mm<SVA<50 mm, standing PT angle<20°, −10°<PI−LL<10° simultaneously), the pelvic posture in the sagittal plane of the patients after hip replacement surgery will not change significantly, that is, a change of the pelvic posterior tilt angle of the patients going through pelvic rebalance is 0;
\textcircled{2} for the hip extension muscle cluster weakness patients (muscle strength<Level 4), the pelvis posture in the sagittal plane of the patients after hip replacement surgery will not change significantly, that is, the change of pelvic posterior tilt angle of the patients going through pelvic rebalance is 0;
\textcircled{3} for the standing sagittal lumbar vertebra hyperextension patients with PI−LL<−10° and SVA<0, in the case that:
A. if fixed flexion deformity of the hip joint <10° and there is any one of the situations such as severe degeneration/spontaneous fusion/surgical fusion in the lumbar vertebra, then the standing pelvic posture of the patients after hip replacement surgery remains unchanged, that is, the change of pelvic posterior tilt angle of the patients going through pelvic rebalance is 0;
B. if fixed flexion deformity of the hip joint >10° and there is no severe degeneration/spontaneous fusion/surgical fusion in the lumbar vertebra, then after the hip replacement surgery (in which the contralateral hip joint is normal or has gone through the hip replacement surgery as well), the change of the pelvic posterior tilt angle of the patients going through pelvis rebalance is 0.25×PI;
\textcircled{4} for standing sagittal imbalance patients with standing SVA>50 mm, in a standing lateral view of the spine-pelvis or full body of the patients, by using a midpoint of a line connecting centers of the bilateral hip joints (i.e., a rotation center of the acetabulum or femur) as a rotation axis and plotting a vertical in accordance to the standard of enabling a balance of the sagittal plane after the surgery (i.e., SVA=50 mm), the change of the pelvic posterior tilt angle of the patients after hip replacement surgery who go through pelvis rebalance is a rotation angle α by which a center of T1 vertebra rotates to the vertical, and the rotation angle α can be provided as a reference for a target implantation angle of a cup prosthesis.

The above embodiments are only used to illustrate the present disclosure, in which the structure, the connection mode, and the manufacturing process of individual components can be changed, and any equivalent modification and improvement based on the technical scheme of the disclosure shall not be excluded from the protection scope of the disclosure.

What is claimed is:

1. A method for predicting sagittal balance state of pelvis after hip replacement surgery, comprising steps of:
1) obtaining evaluation imaging data of full body sagittal posture of a patient before surgery under different body postures, and obtaining fixed flexion deformity information of bilateral hip joints and an evaluation result of functionality of hip extension muscle clusters through physical examination;
2) performing posture analysis on the evaluation imaging data of full body sagittal posture under different body postures obtained to obtain analysis results of degree of sagittal imbalance and severity of hip abduction deformity of the patient before surgery; and
3) predicting pelvic posture rebalance state in a sagittal plane of the patient after hip replacement surgery according to the analysis results of the degree of sagittal imbalance and the severity of hip abduction deformity as well as the evaluation result of functionality of hip extension muscle clusters of the patient before surgery.

2. The method for predicting sagittal balance state of pelvis after hip replacement surgery according to claim 1, wherein in the step 1), when obtaining the evaluation imaging data of full body sagittal posture of the patient before surgery under different body postures, the different body postures obtained include standing on both legs, standing on one leg, sitting, and squatting.

3. The method for predicting sagittal balance state of pelvis after hip replacement surgery according to claim 1, wherein in the step 1), the evaluation imaging data of full body sagittal posture under each body posture at least includes imaging data from cervical spine to middle femur.

4. The method for predicting sagittal balance state of pelvis after hip replacement surgery according to claim 1, wherein in the step 2), performing posture analysis on the evaluation imaging data of full body sagittal posture under different body postures obtained comprises steps of:
2.1) measuring angle and distance parameters in the evaluation imaging data of full body sagittal posture under different body postures, the angle and distance parameters including pelvic tilting, PT, pelvic incidence, PI, lumbar lordosis, LL, sacral vertical axis, SVA, and sacral pelvic angle, SPA; and
2.2) performing, based on the parameters measured and standard reference ranges, an analysis of the sagittal balance state of the patient before surgery to obtain the analysis results of the degree of sagittal imbalance and the severity of hip abduction deformity of the patient before surgery.

5. The method for predicting sagittal balance state of pelvis after hip replacement surgery according to claim 1, wherein in the step 3), predicting pelvic posture rebalance state in a sagittal plane of the patient after hip replacement surgery comprises steps of:
3.1) classifying, based on the analysis result of the degree of sagittal imbalance, the evaluation result of the hip extension muscle clusters and the fixed hip flexion deformity information, patients into standing sagittal balance patients, hip extension muscle cluster weakness patients, standing sagittal lumbar vertebra hyperextension patients, and standing sagittal imbalance patients; and
3.2) predicting, for different types of the patients, the pelvic posture rebalance state in the sagittal plane of the patients after hip replacement surgery.

6. The method for predicting sagittal balance state of pelvis after hip replacement surgery according to claim 5, wherein in the step 3.2), predicting, for different types of the patients, the pelvic posture rebalance state on the sagittal plane of the patients after hip replacement surgery comprises:
\textcircled{1} for the standing sagittal balance patients who meet 0 mm<SVA<50 mm, standing PT angle<20°, −10°<PI−LL<10° simultaneously, a change of pelvic posterior tilt angle of the patients after hip replacement surgery that go through pelvic rebalance is 0;
\textcircled{2} for the hip extension muscle cluster weakness patients whose muscle strength<Level 4, a change of pelvic posterior tilt angle of the patients after hip replacement surgery that go through pelvic rebalance is 0;
\textcircled{3} for the standing sagittal lumbar vertebra hyperextension patients whose PI−LL<−10° and SVA<0, in a case that:
A. if fixed flexion deformity of the hip joint <10° and there is any one of situations of severe degeneration/spontaneous fusion/surgical fusion in lumbar vertebra, then a change of pelvic posterior tilt angle of the patients after hip replacement surgery that go through pelvic rebalance is 0; and
B. if fixed flexion deformity of the hip joint >10° and there is no severe degeneration/spontaneous fusion/surgical fusion in the lumbar vertebra, then a change of pelvic posterior tilt angle of the patients after hip replacement surgery that go through pelvic rebalance is 0.25×PI; and
\textcircled{4} for the standing sagittal imbalance patients with standing SVA>50 mm, in a standing lateral view of spine-pelvis or full body of the patients, by using a midpoint of a line connecting centers of the bilateral hip joints as a rotation axis and plotting a vertical in accordance to a standard of enabling balance state of the sagittal plane after the surgery, calculating and obtaining a rotation angle α by which a center of T1 vertebra rotates to the vertical to serve it as a change of pelvic posterior tilt angle of the patients after hip replacement surgery that go through pelvis rebalance.
